(19) United States
(12) Patent Application Publication
(10) Pub. No.: US 2016/0008575 A1
(43) Pub. Date: Jan. 14, 2016

(54) SLIDABLE VALVE ADAPTOR FOR STEERABLE SHEATH

(71) Applicant: IMRICOR MEDICAL SYSTEMS, INC., Burnsville, MN (US)

(72) Inventors: Scott Kimmel, Roseville, MN (US); Nicholas James Kampa, Eagan, MN (US); Steven R. Wedan, Savage, MN (US); Timothy Allen Pettit, Becker, MN (US); Douglas A. Page, Apple Valley, MN (US)

(21) Appl. No.: 14/844,739

(22) Filed: Sep. 3, 2015

Related U.S. Application Data
(63) Continuation-in-part of application No. 14/106,177, filed on Dec. 13, 2013, now Pat. No. 9,138,561.
Application No. 14/106,177 is a continuation-in-part of application No. 13/819,981, filed on Jan. 30, 2014, now abandoned.
Application No. 14/106,177 is a continuation of application No. PCT/US2013/074331, filed on Dec. 11, 2013.
(60) Provisional application No. 62/157,785, filed on May 6, 2015.
(60) Provisional application No. 61/576,161, filed on Dec. 15, 2011.

(51) Int. Cl. A61M 25/01; A61M 25/00; A61B 5/055

(52) U.S. Cl. CPC A61M 25/0127; A61B 5/055; A61M 25/0147; A61M 25/0075; A61M 25/0136

(57) ABSTRACT

An MR compatible steerable sheath with a slidable valve adaptor is provided. The slidable valve adaptor is configured to maintain the steerable shaft in a proximal position such that there is slack in first and second longitudinal movement wires when the valve adaptor is in a first position, and is configured to remove slack from the first and second longitudinal movement wires when the valve adaptor is slidably moved to the second position. Slidable valve adaptor also optionally includes a safety cap that prevents insertion of a catheter into the control handle until the valve adaptor is in the distal position.

### CROSS REFERENCE TO RELATED APPLICATIONS

This application claims the benefit of priority to U.S. Provisional application Ser. No. 62/157,785, filed on May 6, 2015; and is a continuation-in-part of U.S. application Ser. No. 14/106,177, filed on Dec. 13, 2013; which is a continuation-in-part of U.S. application Ser. No. 13/819,981, filed on Feb. 28, 2013, (abandoned); which claims the benefit of PCT application Serial No.: PCT/US2012/069487, filed on Dec. 13, 2012; which claims the benefit of U.S. Provisional application Ser. No. 61/576,161, filed on Dec. 15, 2011; and U.S. application Ser. No. 14/106,177 is a continuation application of PCT application Serial No.: PCT/US2013/074331, filed on Dec. 11, 2013. The entireties of all of the foregoing are hereby incorporated by reference.

### FIELD OF THE INVENTION

This invention relates to deflectable medical catheters, namely steerable sheaths used in interventional vascular procedures to deliver tools into the human body. More particularly, the present invention is related to a slidable valve adaptor that solves the problems created by sheath lengthening when the sheath is subjected to elevated temperatures.

### BACKGROUND OF THE INVENTION

Deflectable medical catheters, namely steerable sheaths are used in interventional vascular procedures to deliver tools (e.g. electrophysiology catheters, guidewires, balloons catheters, stents, instruments, etc.) into the human body.

MRI has achieved prominence as a diagnostic imaging modality, and increasingly as an interventional imaging modality. The primary benefits of MRI over other imaging modalities, such as X-ray, include superior soft tissue imaging and avoiding patient exposure to ionizing radiation produced by X-rays. MRI's superior soft tissue imaging capabilities have offered great clinical benefit with respect to diagnostic imaging. Similarly, interventional procedures, which have traditionally used X-ray imaging for guidance, stand to benefit greatly from MRI's soft tissue imaging capabilities. In addition, the significant patient exposure to ionizing radiation associated with traditional X-ray guided interventional procedures is eliminated with MRI guidance.

A variety of MRI techniques are being developed as alternatives to X-ray imaging for guiding interventional procedures. For example, as a medical device is advanced through the patient's body during an interventional procedure, its progress may be tracked so that the device can be delivered properly to a target site. Once delivered to the target site, the device and patient tissue may be monitored to improve therapy delivery. Thus, tracking the position of medical devices is useful in interventional procedures. Exemplary interventional procedures include, for example, cardiac electrophysiology procedures including diagnostic procedures for diagnosing arrhythmias and ablation procedures such as atrial fibrillation ablation, ventricular tachycardia ablation, atrial flutter ablation, Wolfe Parkinson White Syndrome ablation, AV node ablation, SVT ablations and the like. Tracking the position of medical devices using MRI is also useful in oncological procedures such as breast, liver and prostate tumor ablations; and urological procedures such as uterine fibroid and enlarged prostate ablations.

MRI uses three fields to image patient anatomy: a large static magnetic field, a time-varying magnetic gradient field, and a radiofrequency (RF) electromagnetic field. The static magnetic field and time-varying magnetic gradient field work in concert to establish both proton alignment with the static magnetic field and also spatially dependent proton spin frequencies (resonant frequencies) within the patient. The RF field, applied at the resonance frequencies, disturbs the initial alignment, such that when the protons relax back to their initial alignment, the RF emitted from the relaxation event may be detected and processed to create an image.

Each of the three fields associated with MRI presents safety risks to patients when a medical device is in close proximity to or in contact either externally or internally with patient tissue. One important safety risk is the heating that may result from an interaction between the RF field of the MRI scanner and the medical device (RF-induced heating), especially medical devices that have elongated conductive structures, such as braiding and pull-wires in catheters and sheaths.

The RF-induced heating safety risk associated with elongated metallic structures in the MRI environment results from a coupling between the RF field and the metallic structure. In this case several heating related conditions exist. One condition exists because the metallic structure electrically contacts tissue. RF currents induced in the metallic structure may be delivered into the tissue, resulting in a high current density in the tissue and associated Joule or Ohmic tissue heating. Also, RF induced currents in the metallic structure may result in increased local specific absorption of RF energy in nearby tissue, thus increasing the tissue's temperature. The foregoing phenomenon is referred to as dielectric heating. Dielectric heating may occur even if the metallic structure does not electrically contact tissue, such metallic braiding used in a steerable sheath. In addition, RF induced currents in the metallic structure may cause Ohmic heating in the structure, itself, and the resultant heat may transfer to the patient. In such cases, it is important to attempt to both reduce the RF induced current present in the metallic structure and/or eliminate it all together by eliminating the use of metal braid and long metallic pull-wires.

The static field of the MRI will cause magnetically induced displacement torque on any device containing ferromagnetic materials and has the potential to cause unwanted device movement. It is important to construct the sheath and control handle from non-magnetic materials, to eliminate the risk of unwanted device movement.

When performing interventional procedures under MRI guidance, clinical grade image quality must be maintained. Conventional steerable sheaths are not designed for the MRI and may cause image artifacts and/or distortion that significantly reduce image quality. Constructing the sheath from non-magnetic materials and eliminating all potentially resonant conductive structures allows the sheath to be used during active MR imaging without impacting image quality. Similarly, it is as important to ensure that the control handle is also constructed from non-magnetic materials thereby eliminating potentially resonsant conductive structures that may prevent the control handle being used during active MR imaging.

MR compatible steerable sheaths utilize a fiber optic braid, a replacement for the stainless steel braid that has traditionally been used in sheath and catheter shafts. The advantage of the fiber optic braid is that it is entirely non-metallic, and therefore MR compatible. In addition, the fiber optic braid still imparts similar mechanical attributes to the sheath shaft as does a stainless steel braid. However, one significant disadvantage of the fiber optic braid is that when it is exposed to elevated temperatures, such as during a sterilization process, it expands in the linear direction and increases the overall length of the sheath shaft. Studies of sheath shaft designs have shown that the shaft may lengthen as much as 0.250″ during the elevated temperatures (65° C.). This effect has also occurs in shafts constructed with non-MR compatible sheaths such as stainless steel braid, but the lengthening is less, about 0.080″. When the shaft returns to room temperature, the length of the shaft returns to is original length. However, the expansion of the shaft creates an issue for the sheath in which the shaft is housed.

During the manufacture of the sheath, the shaft is assembled with taught pull wires. If the shaft is not assembled in this fashion, it creates a ‘dead zone’ in the sheath handle. The ‘dead zone’ is a moment in the sheath handle knob rotation in which movement of the knob causes no deflection in the sheath in either direction. Clinicians are accustomed to a slight ‘dead zone’ but more than half a knob turn is not desirable. Some clinicians, however, have expressed a desire for total elimination of the dead zone.

The sheath is subjected to elevated temperatures during the sterilization process prior to use. Additionally, the sheath assembly may also be exposed to elevated temperatures during transportation and storage as it makes its way to a hospital. When subjected to elevated temperatures the fiber optic braid expands, as noted above, and in turn causes the sheath shaft to expand. Because the pull wires are taught, as assembled, and made of non-expansionable Kevlar, the stress of the expansion has to be relieved somewhere in the shaft. The stress relief location is typically the softest section of the shaft, in which the steerable region is located. This results in permanent compression of the steerable region. When the shaft returns to normal temperature, the permanent deformation in the steerable section creates slack in the pull wires, which results in a significant ‘dead zone’ in the sheath handle.

Thus what is needed is a design MR compatible control handle that solves the foregoing dead-zone issues.

### BRIEF SUMMARY OF THE INVENTION

The foregoing need is addressed by the steerable sheath with slidable valve adaptor in accordance with the invention. Those of skill in the art will appreciate that the valve adaptor in accordance with the invention is disclosed as being utilized with the steerable sheath and control handle as described herein but may also be utilized with other steerable sheaths and control handles, all of which fall within the scope of the invention.

In one aspect of the invention a steerable sheath is provided that may be used in an MRI environment to deliver a variety of tools (catheters, guidewires, implantable devices, etc.) into the lumens of the body.

In a further aspect of the invention, the steerable sheath shaft comprises a reinforced polymer tube in which the reinforcing material is non-metallic based (Kevlar, PEEK, Nylon, fabric, polyimide, etc.) or a hybrid of metallic and non-metallic materials and the reinforcing geometry may comprise a braid, a coil, or a slit tube that mimics a coil and combinations of the foregoing. In yet another aspect of the invention, the reinforced polymer tube may also be segmented with varying flexibility along its length to provide the user with the ability to deflect the catheter in a region in which the segment is more flexible than other segments.

In yet another aspect of the invention the polymer tube may also include one or more passive visualization markers along the length of the tube and/or one or more active visualization markers along the length of the tube.

The steerable sheath in accordance with the invention also includes one or more pull-wires which are coupled with the reinforced tube and that allow the user to manipulate and deflect the polymer tube. In one aspect of the invention, the pull-wires are preferably made of a non-metallic material (Kevlar, PEEK, Nylon, fabric, etc.). One or more internal pull-wire lumens are positioned within the polymer tube construct and allow the user to manipulate the pull-wires to move smoothly during actuation. One or more anchor points connect the pull-wire in the distal portion of the polymer tube.

In another aspect of the invention a control handle on the proximal end of the reinforced tube operates longitudinal movement of the pull-wire(s). In one aspect of the invention, the handle includes paramagnetic or diamagnetic materials or combinations of paramagnetic and diamagnetic materials.

In another aspect of the invention, an MR compatible steerable sheath is provided. The MR compatible steerable sheath includes a steerable shaft including a proximal end and a deflectable distal tip, the steerable shaft configured to receive first and second longitudinal movement wires operably coupled to the deflectable distal tip, the proximal end slidably receivable within a lumen of a t-valve axel; a hemostasis valve assembly operably coupled to the proximal end of the steerable shaft; a slidable valve adaptor operably coupled to the hemostasis valve assembly and configured to be slidably receivable within the lumen of the t-valve axel; a control handle having a main body configured to receive the valve adaptor and hemostasis valve assembly and the first and second rack screws, the second rack screw including a threaded portion on an outer surface thereof, the steerable shaft extending axially through the control handle; the first longitudinal movement wire operably coupled to the first rack screw and the second longitudinal movement operably coupled to the second rack screw; and a rotatable adjustment knob operably engageable with the control handle, the rotatable adjustment knob having an internal threaded portion matingly engageable with the threaded portion of the second rack screw, the rotatable adjustment knob moveable between a first position in which the internal thread is configured to engage the thread on the outer surface of the second rack screw and cause the second rack screw to move proximally to cause proximal longitudinal movement of the second longitudinal movement wire and a second position in which the internal thread is configured to move the second rack screw in a distal direction to release tension on the second longitudinal movement wire, wherein the valve adaptor is configured to remove the slack from the first and second longitudinal movement wires when slidingly moved to a second position.

In another aspect of the invention, a valve adaptor is coupled to the sheath hemostasis valve assembly, the sheath hemostasis valve assembly being coupled to the sheath shaft. The valve adaptor is slidably moveable from a first position to a locked second position.

In another aspect of the invention, the slidable valve adaptor is configured to maintain the steerable shaft in a proximal position such that there is slack in said first and second longitudinal movement wires when said valve adaptor is in a first position, said slidable valve adaptor is configured to remove slack from said first and second longitudinal movement wires when said valve adaptor is slidably moved to said second position.

In another aspect of the invention the distance between the first and second positions is approximately 0.250″ or greater.

In another aspect of the invention a locking mechanism is provided to lock the valve adaptor in the second position.

In another aspect of the invention the valve adaptor moves to the first position by providing a mating relationship between the valve adaptor and a collar on the sheath.

In another aspect of the invention the valve adaptor moves to the first position by providing a spring mechanism that automatically moves the valve adaptor to the first position.

These and other aspects of the invention will now be described in detail with reference to the accompanying Figures.

### BRIEF DESCRIPTION OF THE DRAWINGS

For a better understanding of the invention, and to show how the same may be carried into effect, reference will now be made, by way of example, to the accompanying drawings, in which:

FIG. 1 is a perspective view of a control handle that may be operably coupled with the steerable sheath according to an aspect of the invention.

FIG. 2A is an exploded perspective view of the control handle and steerable sheath according to an aspect of the invention.

FIG. 2B is an exploded perspective view of the control handle and steerable sheath according to another aspect of the invention.

FIG. 2C is an enlarged view of the rotatable adjustment knob including internal threads that are circumferentially disposed about an inner wall thereof.

FIG. 3 is a perspective view of the steerable sheath shaft according to an aspect of the invention.

FIG. 4 is a perspective view of the steerable sheath shaft according to an aspect of the invention with the steerable distal tip cut away to show detail.

FIG. 5A is an enlarged view of the pull wires at the proximal end of the steerable sheath shaft in accordance with the invention.

FIG. 5B is a detailed view of a pull ring that provides a contact point between the pull wire and the distal end of the steerable sheath shaft in one aspect of the invention.

FIG. 6A is a side view of the control handle and steerable sheath of FIG. 2A.

FIG. 6B is a side view of the control handle and steerable sheath of FIG. 2B.

FIG. 7A is an enlarged view of the control handle mechanical structure denoted by 600 in FIG. 6A and showing clockwise rotation of rotatable knob.

FIG. 7B is an enlarged view of the control handle mechanical structure denoted by 600′ in FIG. 6B and showing clockwise rotation of rotatable knob.

FIG. 8A is an enlarged view of the control handle mechanical structure denoted by 800 in FIG. 6A and showing counterclockwise rotation of rotatable knob.

FIG. 8B is an enlarged view of the control handle mechanical structure denoted by 800′ in FIG. 6B and showing counterclockwise rotation of rotatable knob.

FIG. 9A is a side view of the control handle of FIG. 2A showing the function of the pull wire.

FIG. 9B is a side view of the control handle of FIG. 2B showing the function of the pull wire.

FIG. 10A is a perspective view of the control handle and valve adaptor in the proximal position.

FIG. 10B illustrates the valve adaptor of FIG. 10A in the distal position.

FIG. 10C is an exploded view of the control handle and valve adaptor in accordance with the invention with parts omitted.

FIG. 11A is a cut away top view of the control handle with the valve adaptor in the proximal position.

FIG. 11B is an enlarged view of the area labeled 100A of FIG. 11A.

FIG. 12A a cut away top view of the control handle with the valve adaptor in the distal position.

FIG. 12B is an enlarged view of the area labeled 100B of FIG. 12A.

FIGS. 13A, 13B and 13C illustrate the locking mechanism of the slidable valve adaptor in accordance with the invention in the proximal position, intermediate position, and distal locked position.

FIGS. 14A-14B are perspective views of the optional safety cap in accordance with the invention.

FIG. 15A illustrates a cut away view of the safety cap in accordance with the invention positioned within the slidable valve adaptor.

FIG. 15B is an enlarged view of the area marked 1500A of FIG. 15A.

FIGS. 16A-16D are side views of the safety cap in accordance with the invention showing the operation thereof.

### DETAILED DESCRIPTION OF THE INVENTION

Numerous structural variations of an MR compatible steerable sheath and control handle in accordance with the invention are contemplated and within the intended scope of the invention. Those of skill in the art will appreciate that the exemplary control handle may be coupled to other types of steerable sheath shafts. In addition, those of skill in the art will appreciate that the exemplary steerable sheath shaft may be coupled with other control handles. Therefore, for purposes of discussion and not limitation, an exemplary embodiment of the MR compatible steerable sheath shaft and control handle with valve adaptor will be described in detail below.

Referring to the FIGS. like elements have been numbered with like reference numerals.

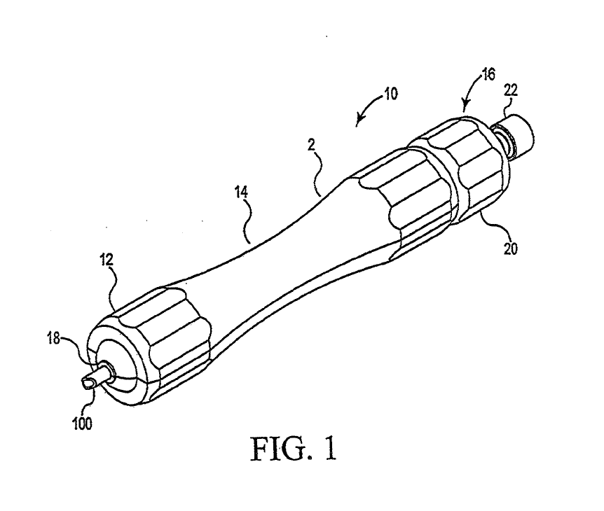

Referring now to FIG. 1, the control handle 10 in accordance with the invention includes a cover 2 as illustrated in FIG. 1. Cover 2 includes distal portion 12, hand-graspable middle region 14, and proximal end 16. Distal portion 12 includes aperture 18 through which steerable sheath shaft 100 exits. Proximal end 16 includes rotatable adjustment knob 20 and port 22. Rotatable adjustment knob 20 is operably coupled to a proximal end (not shown) of steerable sheath shaft 100 such that rotation of the knob causes movement of steerable sheath shaft 100 as hereinafter described. Port 22 includes an aperture therethrough for receiving a medical device such as by way of example an MR-compatible electrode circuit such as that disclosed in U.S. Publn. No. 2011/0046707, the entirety of which is hereby incorporated by reference.

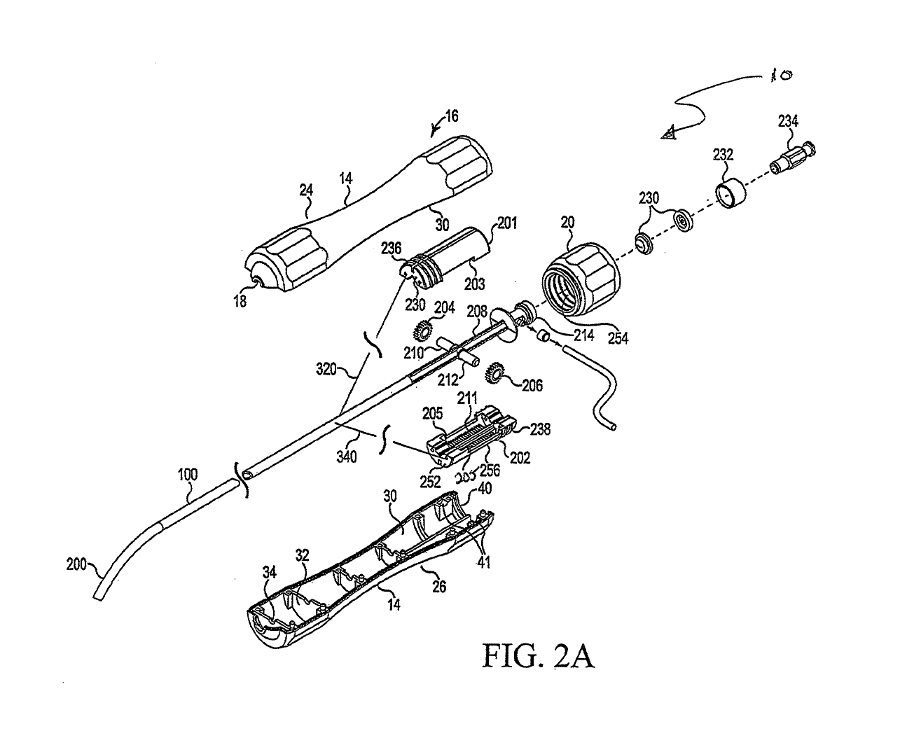

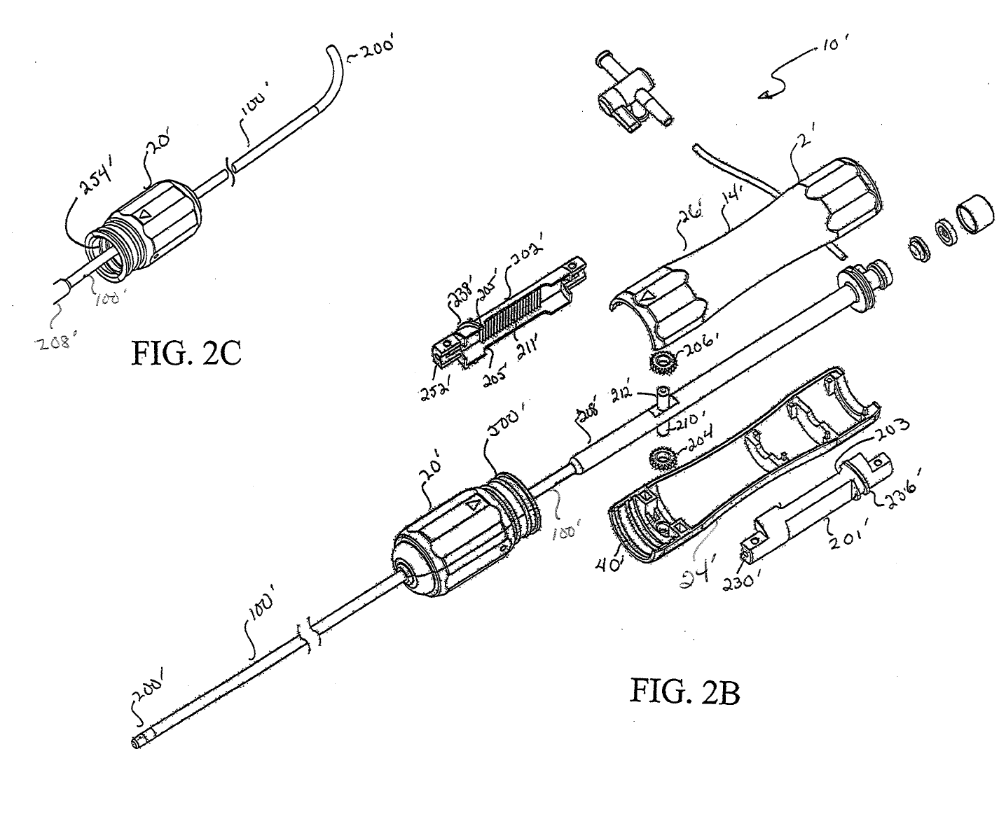

Referring now to FIG. 2A an exploded view of the control handle 10 and steerable sheath shaft 100 in accordance with the invention is shown. Cover 2 of control handle 10 includes a first mating portion 24 and a second mating portion 26. Those of skill in the art will appreciate, however, that cover 2 may include any number of mating portions and still be within the scope of the invention. Each of the first and second mating portions 24, 26 include an inner face 30 having a plurality of inserts 32 fixedly coupled to inner face 30. As depicted, inserts 32 include a receiving groove therewithin. When first mating portion and second mating portion are operably coupled, receiving groove 34 forms a lumen into which steerable sheath shaft 100 is received. First mating portion 24 and second mating portion 26 when mated form an internal recess 40 at a distal end thereof, which accommodates first and second rack screws 201, 202. It should be noted that the distal threads 236 of the first rack screw 201, although shown, have no function. First and second rack screws 201, 202 are simply mirror images of each other and the distal threads 236 of the first rack screw 201 are present to reduce the cost of manufacturing so that first and second rack screws 201, 202 can be made from the same mold. Control handle 10 further includes first and second pinion gears 204, 206, t-valve axel 208, first and second pegs 210, 212, t-valve 214, tube retainer 216, tube 218, and rotatable adjustment knob 20. Rotatable adjustment knob 20 receives seals 230, seal cap 232 and fitting 234. First and second pegs 210, 212 are operably coupled to t-valve axel 208. Groove 41 receives pegs 210, 212. First and second pegs 210, 212 receive pinion gears 204 and 206. Tube 218 attaches to a stopcock in t-valve which connects to a syringe for flushing or aspirating the steerable catheter.

As may be seen in FIG. 2A, second rack screw 202 includes proximal threads 238 on an outer surface thereof. Those of skill in the art will appreciate that “first” and “second” rack screws are relative terms. Those of skill in the art will also appreciate that the control knob 20 may be positioned distally to first and second rack screws and the orientation of first and second rack screws flipped as will be described below with reference to FIG. 2B. An internal central channel of each of first and second rack screws 201, 202 includes a threaded portion 211 that threadably receives pinion gears 204, 206 in operation. First and second rack screws 201, 202 include notched portion 203, 205. First and second pull wires 320, 340 are routed and are operably coupled to ends 230, 252 of each rack screw 201, 202, respectively. Pinion gears 204, 206 are received by pegs 210, 212 operably coupled to t-valve axel 208. T-valve axel 208 is bonded to sheath shaft 100. In operation, posts 210, 212 are received by and move longitudinally on notched portion 203, 205 respectively. This allows threaded pinion gears 204, 206 to be received by and move longitudinally along the threaded central channel of each of first and second rack screws 201, 202.

As seen in FIG. 2A, rotatable adjustment knob 20 includes internal threads 254 circumferentially disposed about an inner wall thereof. Internal threads 254 will engage the proximal threads 238 of the second rack screw 202. As the rotatable adjustment knob is rotated clock-wise the internal adjustment knob threads 254 engage the proximal threads 238 of the second rack screw 202 causing longitudinal, proximal movement of rack screw 202. As the rotatable adjustment knob is rotated counter-clockwise the internal threads (still engaged with the proximal threads 238 of the second rack screw 202) causes longitudinal, distal movement of rack screw 202.

Those of skill in the art will appreciate that the orientation of the first and second rack screws may be changed without departing from the scope of the invention. As may be seen in FIG. 2B, second rack screw 202′ includes distal threads 238′ on an outer surface thereof. An internal central channel of each of first and second rack screws 201′, 202′ includes a threaded portion 211′ that threadably receives pinion gears 204′, 206′ in operation. First and second rack screws 201′, 202′ include notched portion 203′, 205′. First and second pull wires (not shown) are routed and are operably coupled to ends 230′, 252′ of each rack screw 201′, 202′, respectively. Pinion gears 204′, 206′ are received by pegs 210′, 212′ operably coupled to t-valve axel 208′. T-valve axel includes a lumen therewithin that slidably receives sheath shaft 100′ at a distal end thereof. In operation, posts 210′, 212′ are received by and move longitudinally on notched portion 203′, 205′ respectively. This allows threaded pinion gears 204′, 206′ to be received by and move longitudinally along the threaded central channel of each of first and second rack screws 201′, 202′.

As seen in FIG. 2C, rotatable adjustment knob 20′ includes internal threads 254′ circumferentially disposed about an inner wall thereof. Internal threads 254′ will engage the distal threads 238′ of the second rack screw 202′. As the rotatable adjustment knob 20′ is rotated clock-wise the internal adjustment knob threads 254′ engage the distal threads 238′ of the second rack screw 202′ causing longitudinal, proximal movement of rack screw 202′. As the rotatable adjustment know is rotated counter-clockwise the internal threads (still engaged with the distal threads 238′ of the second rack screw 202′) causes longitudinal, distal movement of rack screw 202′. Thus, those of skill in the art will appreciate that although the rotatable adjustment knob 20′ is positioned distal to the first and second rack screws 201′, 202′ the operation of the control handle has not changed.

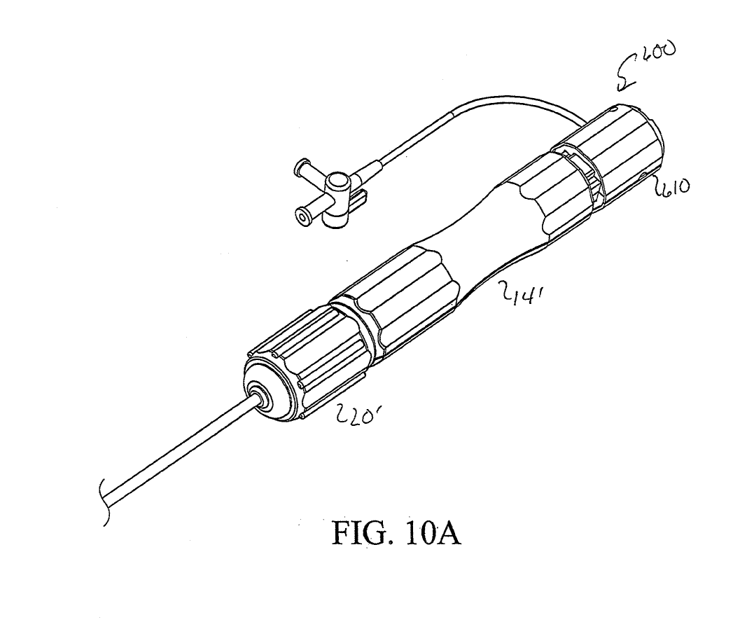

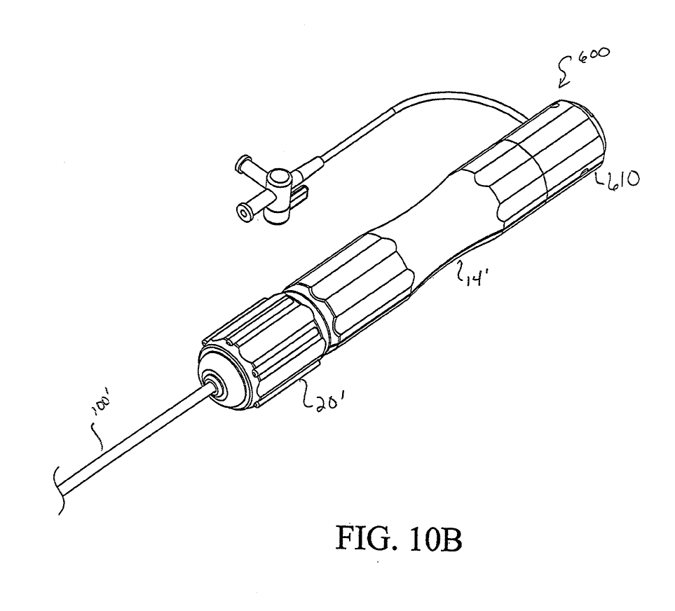

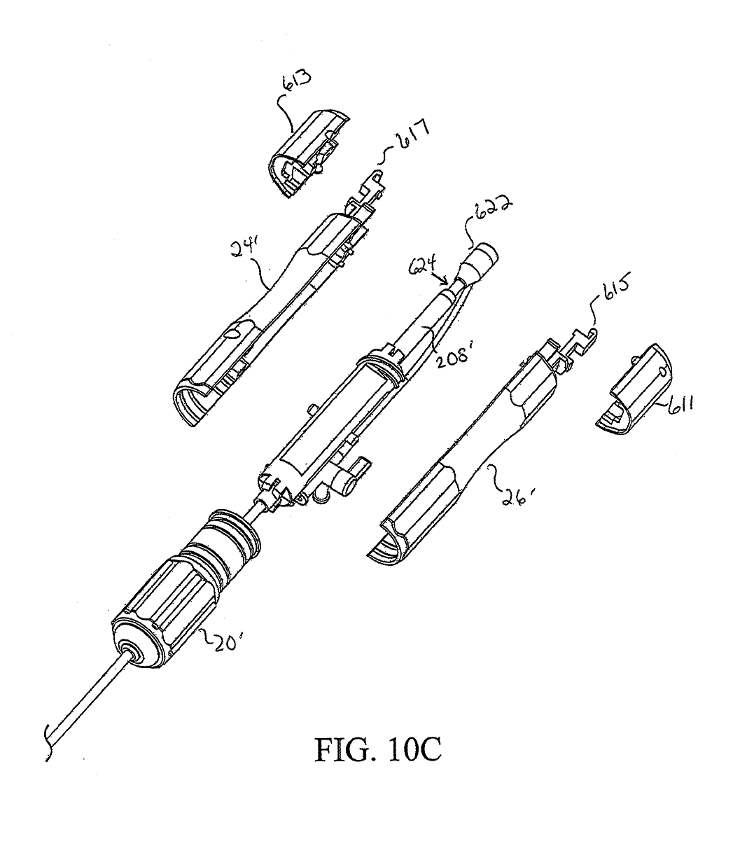

Rotatable adjustment knob 20′ of FIGS. 2B and 2C includes grooves 500 on an outer surface thereof which, in operation, accommodate a plurality of O-rings 510 (as best seen in FIG. 10) that create a friction fit between the knob 20′ and the first mating portion 24′ and second mating portion 26′ of cover 2 of control handle 10, which has corresponding grooves.

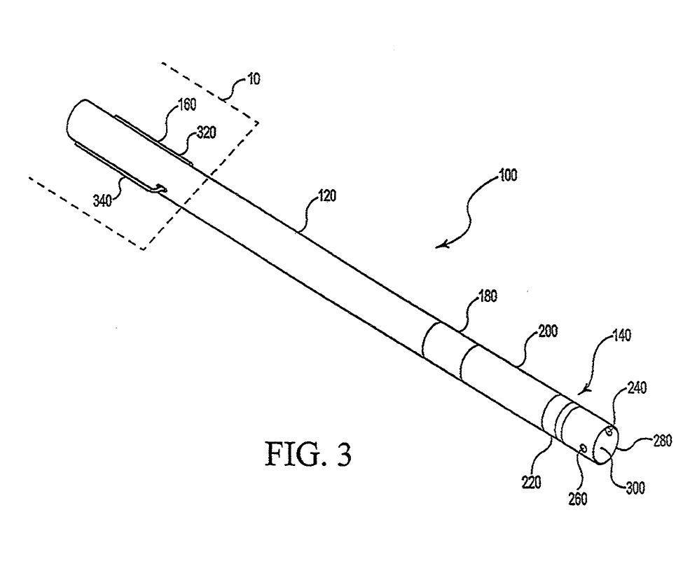

Referring now to FIG. 3, the steerable sheath shaft 100 in accordance with the invention will now be explained. Steerable sheath shaft 100 may be used in an MRI environment to deliver a variety of tools such as catheters, guide wires, implantable devices, etc. into cavities and passageways of a patient body. The steerable sheath shaft 100 includes a deflectable tip portion 200 that is able to bend at least 180 degrees offset from the longitudinal axis of the catheter sheath shaft 100. This flexibility allows the medical professional to make very tight turns to deliver the aforementioned tools to the cavities and passageways of the patient body.

Referring again to FIG. 3 a perspective view of an MR compatible steerable sheath that is suitable for use in an MRI environment is depicted. The MR compatible steerable sheath shaft 100 in accordance with the invention broadly includes tubular shaft 120 with distal 140 and proximal ends 160. Tubular shaft 120 includes an outer diameter 130, an inner diameter 150 and defines a central lumen 300 therewithin. Tubular shaft may be constructed of a variety of polymers such as pebax, polyurethane, nylon, derivatives thereof and combinations of the foregoing.

Distal end 14 includes transition section 180, deflectable tip portion 200, and magnetic marker 220. Pressure relief holes 240, 260 may be formed in the tubular shaft 120 at the distal end 140. Those of skill in the art will appreciate that while only two pressure relief holes 240, 260 are shown there may any number of pressure relief holes formed and still be within the scope of the invention. When retracting an item housed by the sheath shaft 100, such as a catheter or MR active tracking system, pressure may form at the end of the sheath thereby drawing or sucking in tissue. Pressure relief holes 240, 260 are designed to reduce this pressure thereby ameliorating the risk of tissue damage.

Transition section 180 is optionally included for purposes of manufacturability. The deflectable tip section 20 has a significantly lower durometer making it more malleable and flexible than the main body portion 170 of tubular shaft 120 which has a higher durometer or, in other words, quite stiff. As a consequence, these two sections do not bond to one another well. Transitional section 180 has a mid-range durometer allowing it to bond well to both the deflectable tip section 200 and the main body 170 of the tubular shaft 120. Those of skill in the art will appreciate that the transition section 180 may be of any length desired so as to provide an adequate transition between the distal tip portion 200 and the main body portion 170. In one exemplary embodiment transition section may range from about 0.25 to about 0.75 inches. In addition, those of skill in the art will appreciate that transition section may be eliminated and the deflectable tip section 200 may be coupled to the main body 170 of tubular shaft 120 by means known to those of skill in the art without departing from the spirit of the invention.

Steerable sheath shaft 100 includes central lumen 300 therewithin. In one aspect of the invention, the inner diameter 150 of the tubular shaft 120 is approximately 6 French or greater but those of skill in the art will appreciate that varying internal diameters may be used depending on the particular application without departing from the scope of the present invention. Central lumen 300 may include one or more liners (not shown) disposed therewithin to allow for easier movement of instruments therethrough. Liners may comprise materials made from polytetrafluoroethylene (PTFE), fluorinated ethylene propylene copolymer (FEP), nylons and combinations of the foregoing. Alternatively, the lumen 300 may be coated with any such polymers. The polymer tubular shaft 120 may also include one or more passive visualization markers, such as a ferrous or magnetic marker 220, disposed circumferentially about the tubular shaft 120 at one or more locations along the length thereof and/or one or more active visualization markers such as an active tracking coil along the length of the tube. An active tracking coil may comprise one or more small antennas integrated into the device and include traces on a circuit board, coiled wire, and/or a dipole. If an active visualization marker is used, one or more devices may be included in the conductors to mitigate RF field heating may be included. Such devices include chokes, transformers, impedances, and other such devices known to those of skill in the art. One or more fluoroscopy markers (not shown) may also be included along the length of the polymer tubular shaft 12.

One or more optional fluid ports (not shown) may be located on the proximal end 16 of the tubular shaft 12 to allow for homeostasis of the sheath with the patient body. The fluid port(s) allows access for the user or physician to aspirate blood from the steerable sheath lumen 30 and flush with saline. Aspirating and flushing of the sheath prevents air from entering the body before and during insertion of a tool and/or catheter.

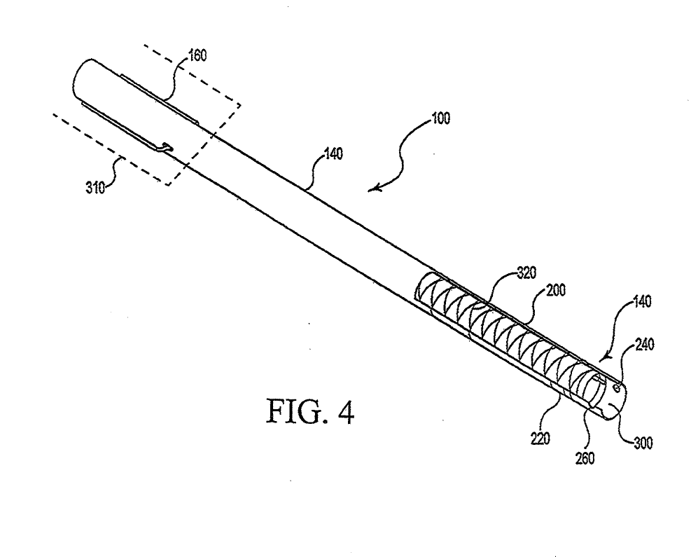

Referring now to FIG. 4 a cut away view of the steerable sheath shaft 100 in accordance with the invention depicts a reinforcement construct 320 of the tubular shaft 120. As shown, the geometry of the reinforcement construct 320 is braided but those of skill in the art will appreciate that the reinforcement construct 320 may comprise other configurations so long as it imparts the necessary deflectability to the tubular shaft 120 at the distal end. For example the reinforcement geometry may be a coil or a slit tube that mimics a coil or combinations of the foregoing. The reinforcement of the tubular shaft 120 may extend from the distal end 140 to the proximal end 160 or may extend from the deflectable tip section 200 to approximately the transition section 180 of the tubular shaft 12.

The material used in the reinforcement construct 320 may be non-metallic such as Kevlar, PEEK, Nylon, fabric, polyimide, fiber optic, silica glass and the like or may also be hybrid of metallic, such as stainless steel, and non-metallic materials. Those of skill in the art will appreciate that, the reinforced polymer tubular shaft 140 may be segmented and each segment may be constructed with varying flexibility along the segment to provide the user with the ability to deflect the sheath in a region in which the segment is more flexible than in other segments. Varying flexibility and thus deflectability may be accomplished by having braids or coils that have greater braiding or coils per sq. cm than in other segments where the braiding or coiling would be less per sq. cm. Flexibility and deflectability may also be accomplished by the varying durometers as herein described.

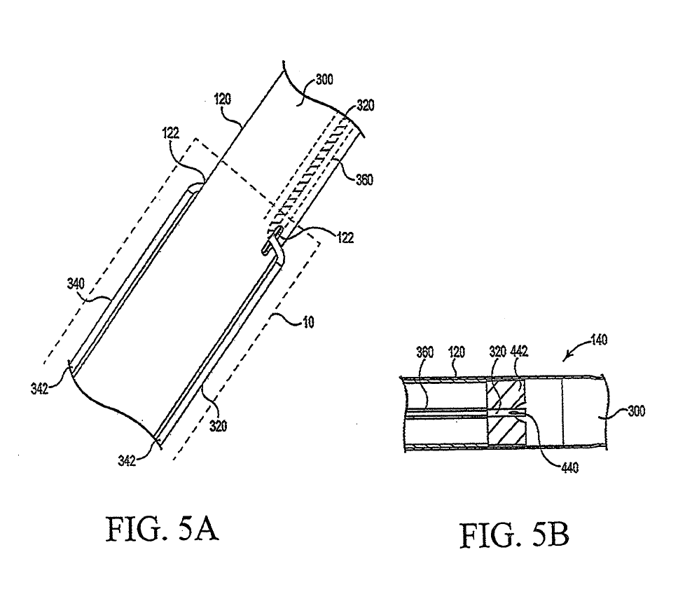

Referring now to FIG. 5A, an enlarged view of the proximal end 160 of the steerable sheath shaft 100 in accordance with the invention is depicted. Proximal end 160 of the steerable sheath is operably coupled to control handle 10 depicted in dashed lines and as hereinafter described. The steerable sheath shaft 100 in accordance with the invention includes one or more pull-wires 320, 340 which are operably coupled at a pull-wire proximal end 342 to the control handle 10 as hereinafter will be described. The portion of the pull-wires 320, 340 that are operably coupled to the control handle exit the tubular body 120 at opening 122. The portion of the pull-wires 320, 340 that are operably coupled to pull ring 440 (as best seen in FIG. 5B) extend through a lumen constructed from a sheet of polymeric material fastened to an inner portion of tubular shaft 120 for a length thereof and enter tubular shaft 120 through entrance holes 330, 350 on opposing sides of tubular shaft 120. Pull-wires 320, 340 allow the user to manipulate and deflect the one or more flexible segments along the length of the polymer tubular shaft 120 and in particular the deflectable tip portion 200. In one aspect of the invention, the pull-wires 320, 340 are preferably made of a non-metallic material (Kevlar, PEEK, Nylon, fabric, etc.).

One or more internal pull-wire lumens 360 are constructed of a flexible, non-metallic material such as PTFE. Internal pull-wire lumens 360 facilitate smooth manipulation of the pull-wires 320, 340 during actuation. Internal pull-wire lumens 360 have an outer diameter of approximately 0.12 inches and an inner diameter of approximately 0.010 inches. However, those of skill in the art will appreciate that the dimensions of the internal pull-wire lumens 360 may vary with the dimensions of both the pull-wires 320, 340 and the tubular shaft 120 so long as they are dimensioned to house the pull-wires and allow pull-wires to move smoothly during actuation.

Referring to FIG. 5B, a side view of the distal end of the steerable sheath in accordance with the invention is shown. Pull wires 320, 340 are operably coupled at their distal end to an opening 440 in pull ring 442 positioned within lumen 300 at the deflectable tip 200 end of the steerable sheath shaft 100.

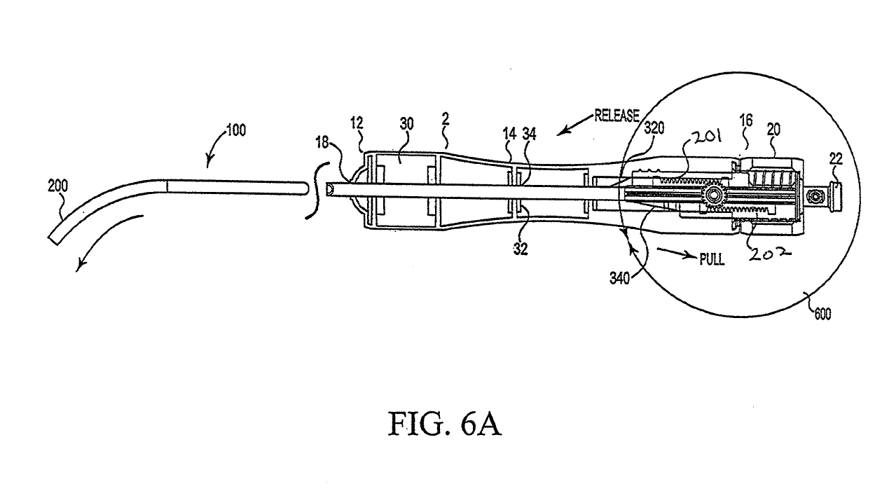

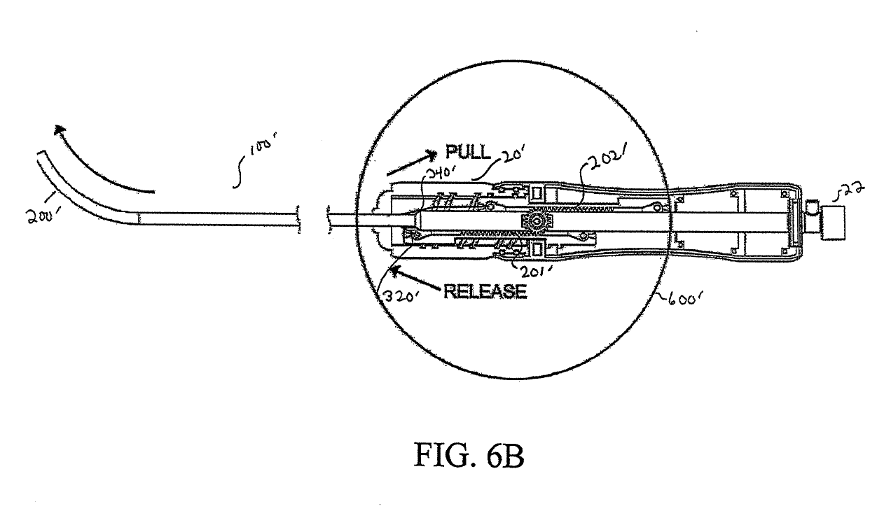

Referring now to FIGS. 6-9 an exemplary control handle 31 for operating the steerable sheath is disclosed. As discussed in reference to FIG. 2, control handle 310 allows the user to control the longitudinal movement of pull-wires 320, 340 which in turn “pull” or deflect the distal end 140 of the steerable sheath shaft 100 in opposite directions. Control handle 310 is positioned on the proximal end of the steerable sheath shaft 100 and operates longitudinal movement of the pull-wire(s) and correspondingly, directional movement of the steerable sheath shaft 100. In one aspect of the invention, control handle 310 includes paramagnetic or diamagnetic materials or combinations of paramagnetic and diamagnetic materials.

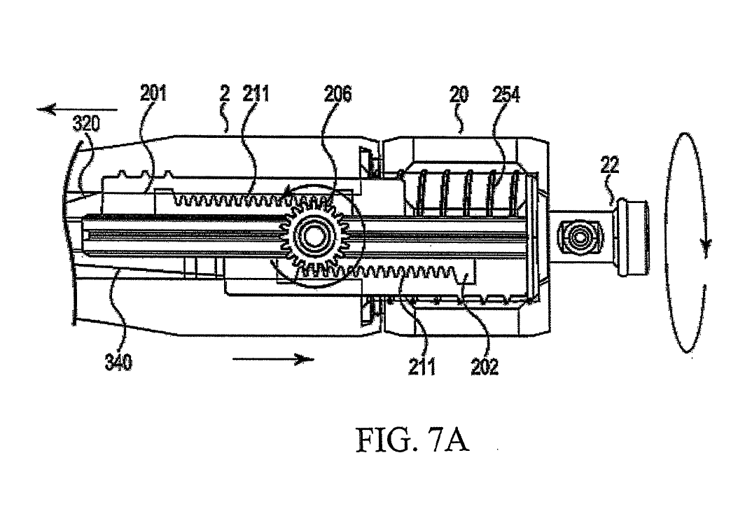

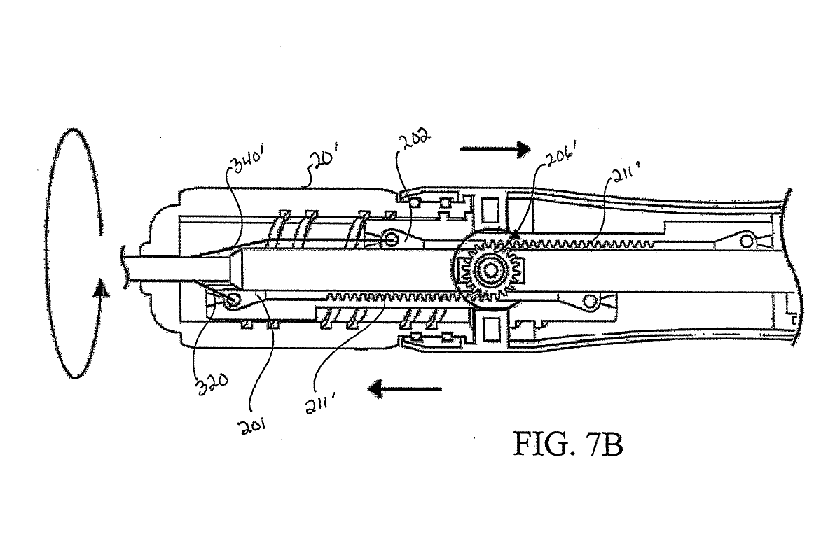

Referring now to FIGS. 6A-7B, FIGS. 7A and 7B are enlarged views of the control handle of FIGS. 6A and 6B denoted at numeral 600, 600′. Adjustment knob 20, 20′ is rotated in the clockwise direction, which causes internal threads 254, 254′ to engage threads 238, 238′ of second rack screw 202, 202′ and cause longitudinal, proximal movement of the second rack screw 202, 202′. At the same time, the pinion gears are engaged by the longitudinal movement of the second rack screw 202, 202′. This causes the first rack screw 201, 201′ to move in the opposite direction, i.e. distally. Distal movement of the first rack screw 201, 201′ releases tension in the first pull wire 320, 320′.

As rotatable adjustment knob 20, 20′ is rotated in the clockwise direction and engages rack screws which in turn engage pinion gears, second pull wire 340, 340′ is pulled toward the proximal direction as best seen in FIGS. 6A and 6B. In turn, the tension on first pull wire 320, 320′ is released. As second pull wire 340, 340′ is pulled in the proximal direction deflectable tip moves in one direction, shown as a downward direction in FIG. 6A and an upward direction in FIG. 6B; however those of skill in the art will appreciate that the direction of deflectable tip is relative to how or the direction in which the user is holding the handle 10. When the t-valve pegs 210, 210′, 212, 212′ abut stops 205, 205′ in second rack screw 202, 202′ the rack screw 202, 202′ stops moving and further movement of rotatable adjustment knob 20, 20′ is halted.

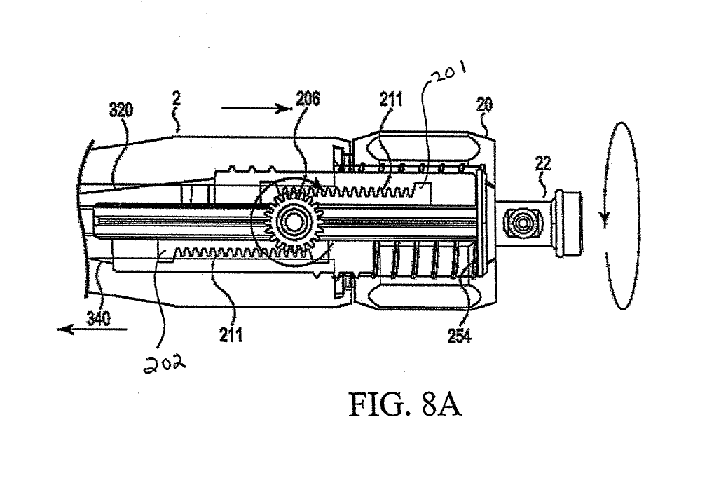

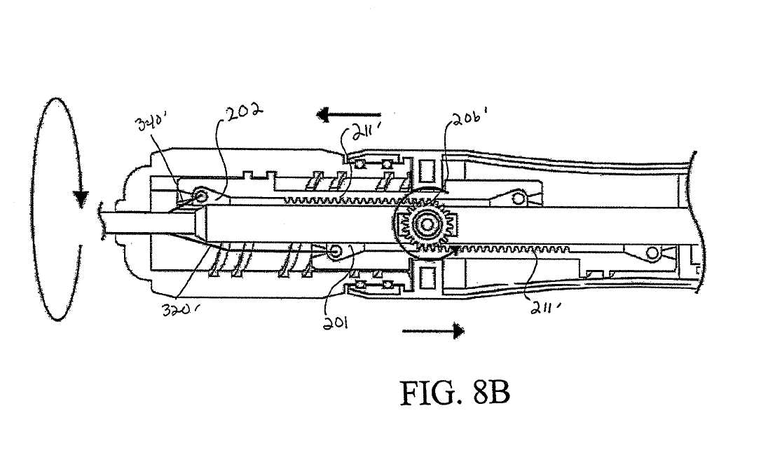

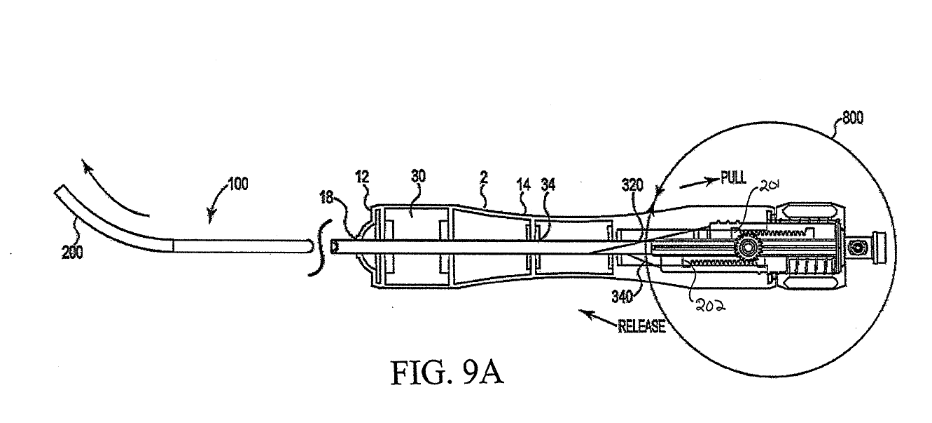

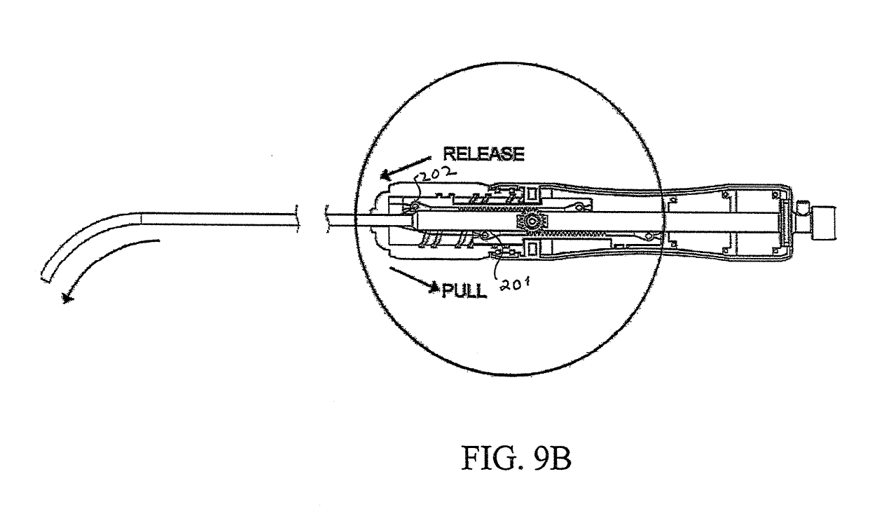

Referring now to FIGS. 8A, 8B and 9A, 9B the opposite function is illustrated. Adjustment knob 20, 20′ is rotated in the counter-clockwise direction, internal threads 254, 254′ engage threads 238, 238′ of second rack screw 202, 202′ causing longitudinal, distal movement. As the rotatable adjustment knob 20, 20′ continues to be rotated in a counter-clockwise direction, pinion gears 204, 204′, 206, 206′ once again operably engage threaded portion 211, 211′ of first and second rack screws.

As rotatable adjustment knob 20, 20′ is rotated in the counter-clockwise direction first pull wire 320, 320′ is pulled toward the proximal direction as best seen in FIGS. 9A and 9B. In turn, the tension on second pull wire 340, 340′ is released. As first pull wire 320, 320′ is pulled in the proximal direction deflectable tip moves in the opposite direction, shown as an upward direction in FIG. 9A and a downward direction in FIG. 9B; however those of skill in the art will appreciate that the direction of deflectable tip is relative to how, or the direction in which, the user is holding the handle 10. When the t-valve pegs 210, 210′, 212, 212′ abut stops 205, 205′ in second rack screw 202, 202′ the rack screw 202, 202′ stops moving and further movement of rotatable adjustment knob 20, 20′ is halted.

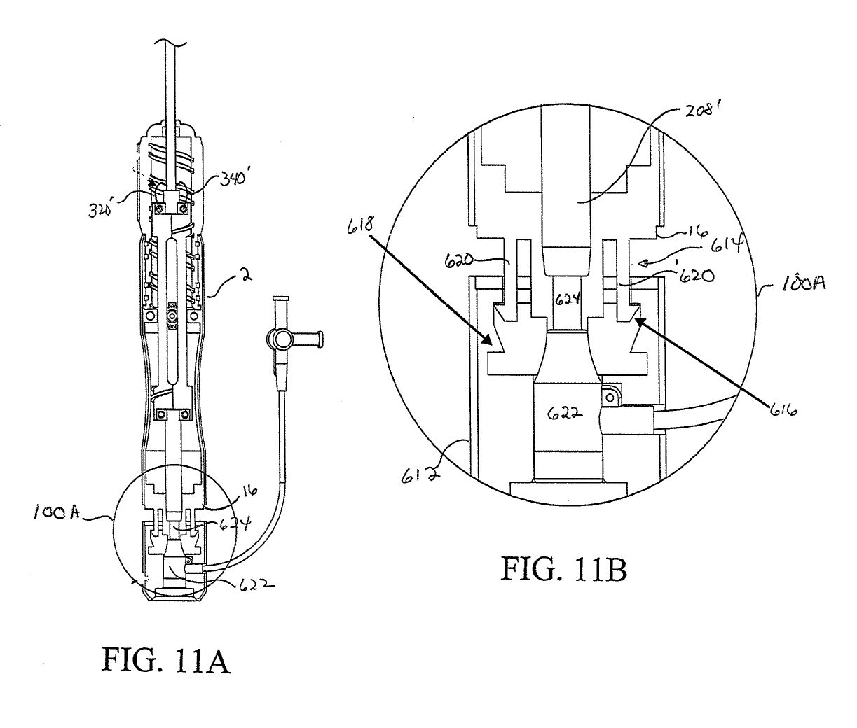

Referring now to FIGS. 10-11, the control handle and steerable sheath shaft of FIGS. 1-9 has been modified to include a valve adaptor 500 in accordance with the invention. An exemplary embodiment will use control handle 10′ of FIG. 2B to describe the invention. As mentioned previously, slack in the pull wires 320′, 340′ results from the device being subjected to elevated temperatures during the sterilization process or during transportation and storage. When subjected to elevated temperatures the fiber optic braid expands, as noted above, and in turn causes the sheath to expand. Because the pull wires are taught, as assembled, and made of non-expansionable Kevlar, the stress of the expansion has to be relieved somewhere in the shaft. The stress relief location is typically the softest section of the shaft, in which the deflectable region is located. This results in permanent compression of the deflectable region. When the shaft returns to normal temperature, the permanent deformation in the deflectable section creates slack in the pull wires, which results in a significant ‘dead zone’ in the sheath handle.

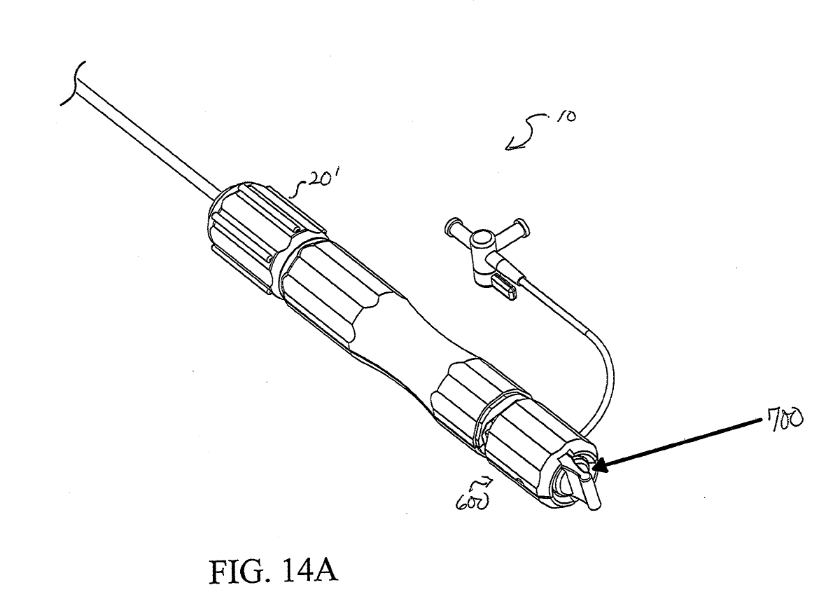

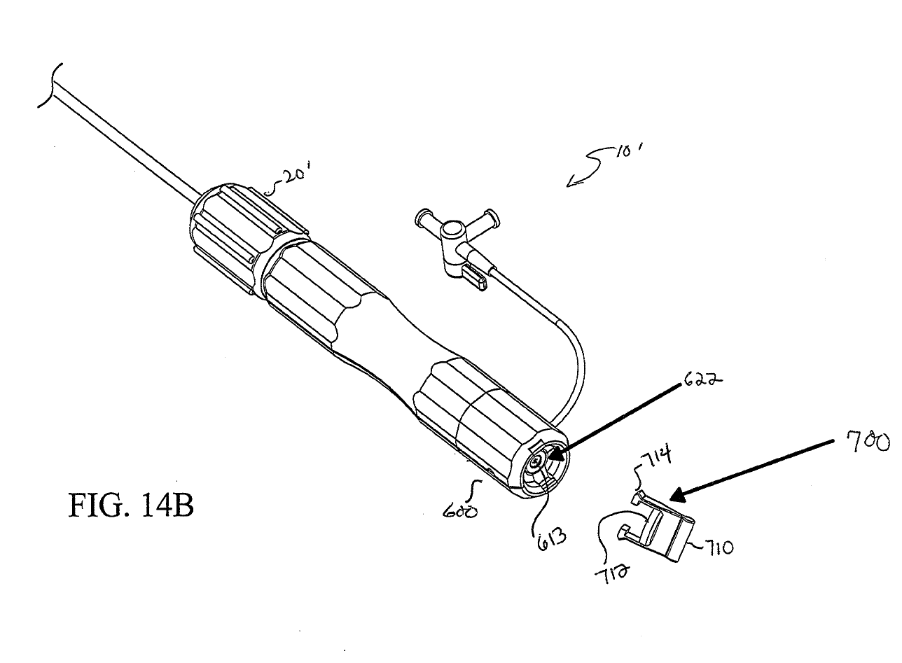

Referring now to FIGS. 10A-12B a valve adaptor 600 in accordance with the invention that overcomes the forgoing issue is illustrated. Valve adaptor 600 broadly includes slidable proximal end piece 610, locking mechanism 614 and optional safety cap 700 (as best seen in FIGS. 14A-17B). As best seen in FIGS. 10B and 10C, slidable proximal end piece 610 includes first 611 and second 613 mating halves. Locking mechanism 614 (as best seen in FIG. 11B) broadly includes first and second mating halves 615, 617 and locking barb 618. Each of first and second mating halves 615, 617 include snap hook 616 and axially extending shaft 620. First 611 and second 613 mating halves include locking barb 618. First and second mating halves 615, 617 are fixedly coupled to mating portions 26′ and 24′ respectively. Proximal end piece 610 is fixedly coupled to valve 622 that is fixedly coupled to the proximal end of steerable shaft 100′ which is slidingly receivable within the lumen oft-valve axel 208′ such that proximal end piece 610, valve 622 and steerable shaft 100′ all move together.

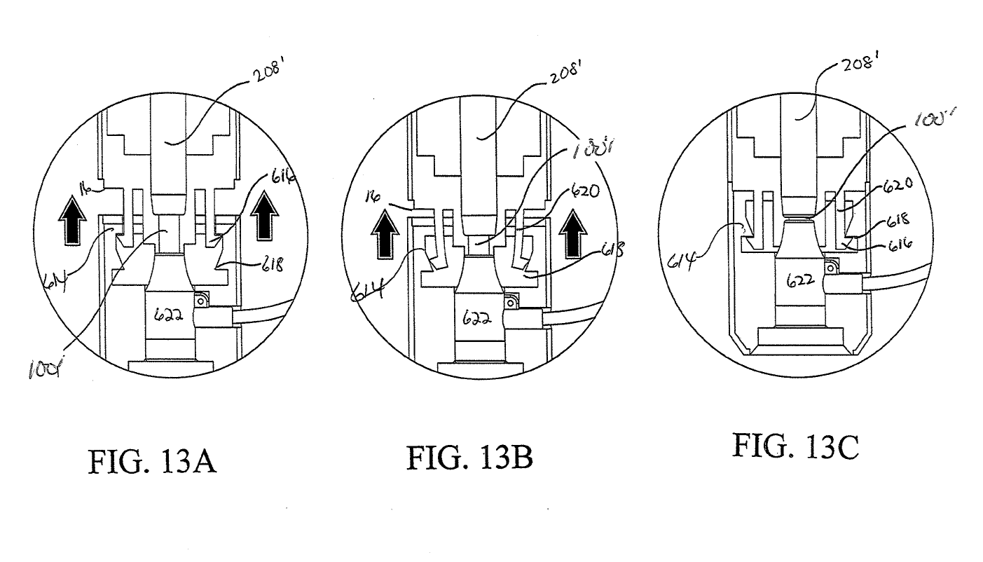

As best seen in FIGS. 11A-13C hemostasis valve 622 is bonded to the proximal end of sheath shaft 100. Shaft 100 is slidably received within a lumen (not shown) oft-valve shaft 208′ with valve adaptor 600 acting as a shaft anchor. Thus valve adaptor 600 is slidably moveable from a first position shown in FIG. 13A to a second position as shown in FIG. 13C in relation to control handle 10′. First and second positions may be proximal and distal. If a sheath is disposable, the valve adaptor 600 may include locking mechanism 614 that locks it into place in the distal position as seen in FIG. 13C. If a sheath is reusable, the valve adaptor 600 may be slidably moveable between a first position as shown in FIG. 13A and a second position as shown in FIG. 13C by eliminating locking mechanism 614.

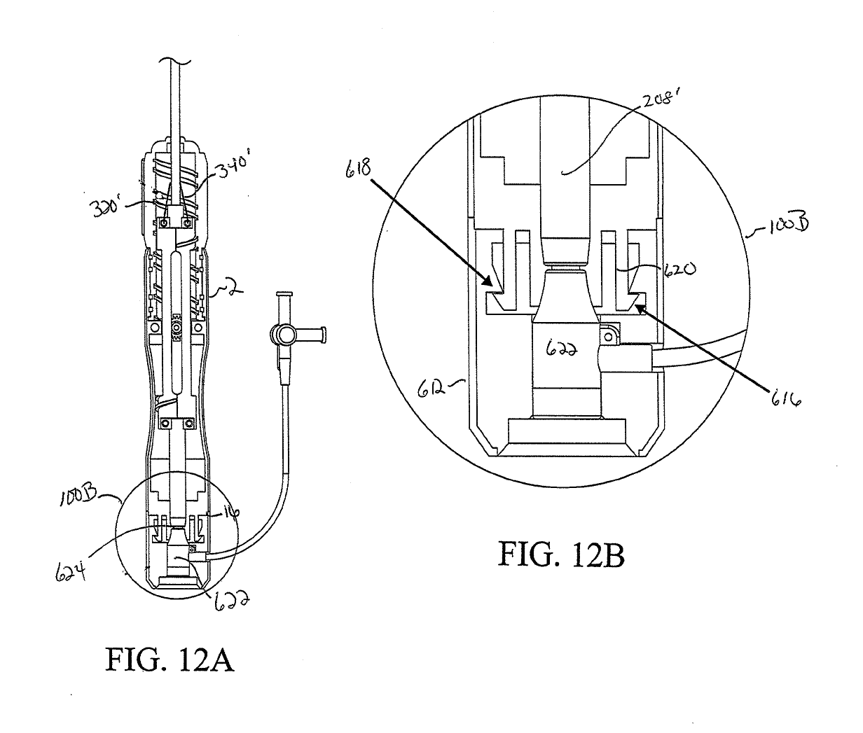

Referring now to FIGS. 11A-12B pull wires 320′, 340′ are operably coupled to the distal ends of first 201′ and second 202′ rack screws, respectively. When the valve adaptor is in the proximal position, the sheath shaft is proximally located in relation to the control handle. This position creates slack in the pull wires 320′, 340′ as best seen in FIG. 11A. When the slidable valve adaptor 600 is slidablely moved to the distal position, the sheath shaft 100 moves distally in relation to the control handle. In this position, the pull wires become taught as best seen in FIG. 12A.

FIGS. 11A-13C illustrate how moving the slidable valve adaptor 600 in a distal direction removes the slack from pull wires 320′, 340. The control handle 10′ in accordance with the invention is assembled and packaged with the valve adaptor 600 in the proximal position. In operation, when the user moves the valve adaptor into the distal position, the sheath shaft 100 slides distally through the lumen of t-valve axel 208′. Those of skill in the art will appreciate that valve adaptor 600 may be moved into the distal position manually or by automated means as hereinafter described. Valve adaptor 600 moves in relation to the t-valve axel 208′ and rack screws 201′, 202′ to remove the slack from the pull wires. The valve adaptor causes the entire sheath shaft 100 to move distally in relation to all the elements of the control handle, including the rack screws and t-valve, so this does cause the rack screws to move proximally in a sense in relation to the sheath shaft.

A critical element of this design is that the distance between the first and second valve adaptor positions must be greater than the largest amount of lengthening the shaft will undergo. In other words, there has to be so much slack that the pull wires never become taught during elevated temperatures and before the valve adaptor is slid into the distal position. Thus, if the shaft undergoes a maximum of 0.250″ of lengthening, then the distance between the first and second valve adaptor positions must be greater than 0.250″.

After the valve adaptor 600 is moved into the distal position, locking mechanism 614 locks it into place when snap hooks 616 engage locking barbs 618. Those of skill in the art will appreciate that many different locking mechanisms may be used including snap hooks, annular snap features, detents, magnets, living hinge hooks, and the like.

Those of skill in the art will appreciate that various manual means for slidably moving the valve adaptor to the distal position fall within the scope of the invention. For example, another manual mechanism may include providing a threaded collar between the valve adaptor and main handle components. In this aspect, the valve adaptor may include a thread on its outer surface that matingly engages a corresponding thread on the collar. When the collar is rotated from a first position to a second position, the threading is such that the valve adaptor moves from the proximal position to the distal position.

In another aspect of the invention, the valve adaptor may move automatically by automatic mechanisms such as a spring. In this aspect, the spring is compressed during packaging. When the sheath is removed from the tray or other packaging, the spring releases and the valve adaptor automatically moves distally. In another aspect, a temperature sensitive piece may be provided. The temperature sensitive piece may comprise Nitinol or other self-expanding materials known to those of skill in the art. The temperature sensitive piece pushes the valve adaptor into the proximal position when the temperature is elevated, but returns the valve adaptor into the distal position when the temperature returns to baseline. This design would be slightly different because the handle would be assembled and packaged such that the valve adaptor is in the distal position.

An optional aspect of the valve adaptor 600 in accordance with the invention includes a safety cap as best seen in FIGS. 14A-14F. The safety cap ensures the valve adaptor 600 is slidably moved distally before the valve lumen 613 and sheath shaft 100 lumen may operably receive a catheter. FIG. 14A illustrates the safety cap in the locked position and FIG. 14B illustrates the safety cap now removable from the valve adaptor 600 in the distal position.

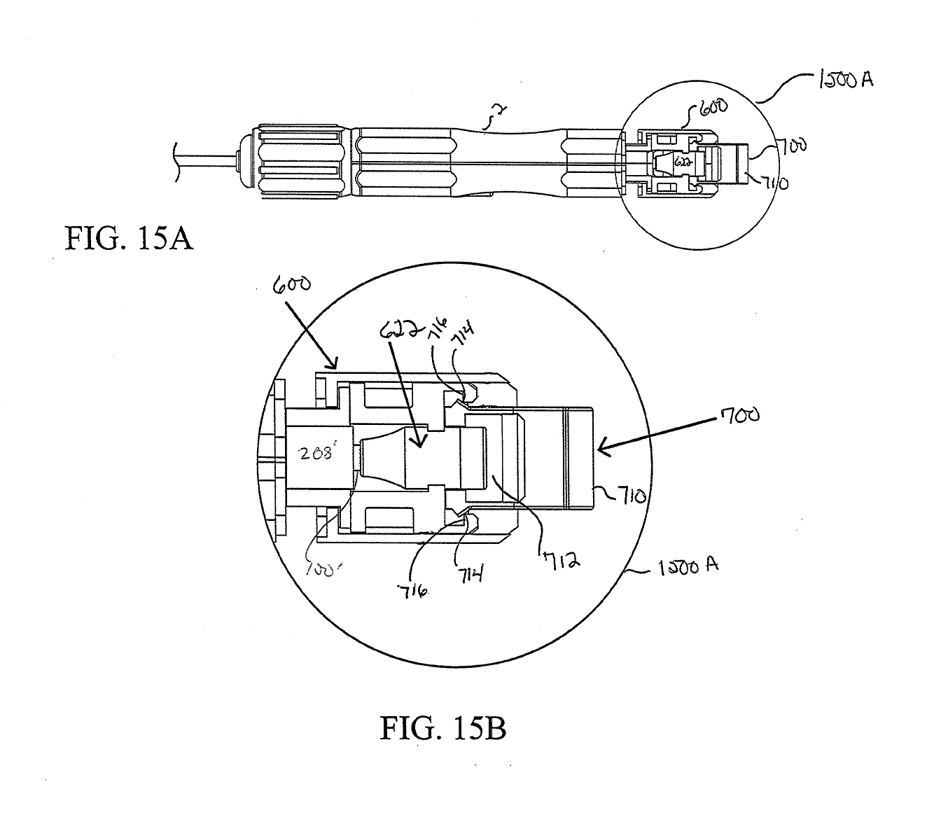

Referring now to FIGS. 14A through 15B safety cap 700 broadly includes finger-graspable end portion 710, blocking element 712 and resilient safety cap hooks 714. Blocking element 712 of safety cap 700 covers the hemostasis valve lumen 613 which operably couples with the sheath shaft 100 lumen when the valve adaptor 600 is in the proximal position as best seen in FIGS. 14A and 15A. Blocking element 712 prevents insertion of a catheter into lumen 613 and sheath shaft 100 lumen.

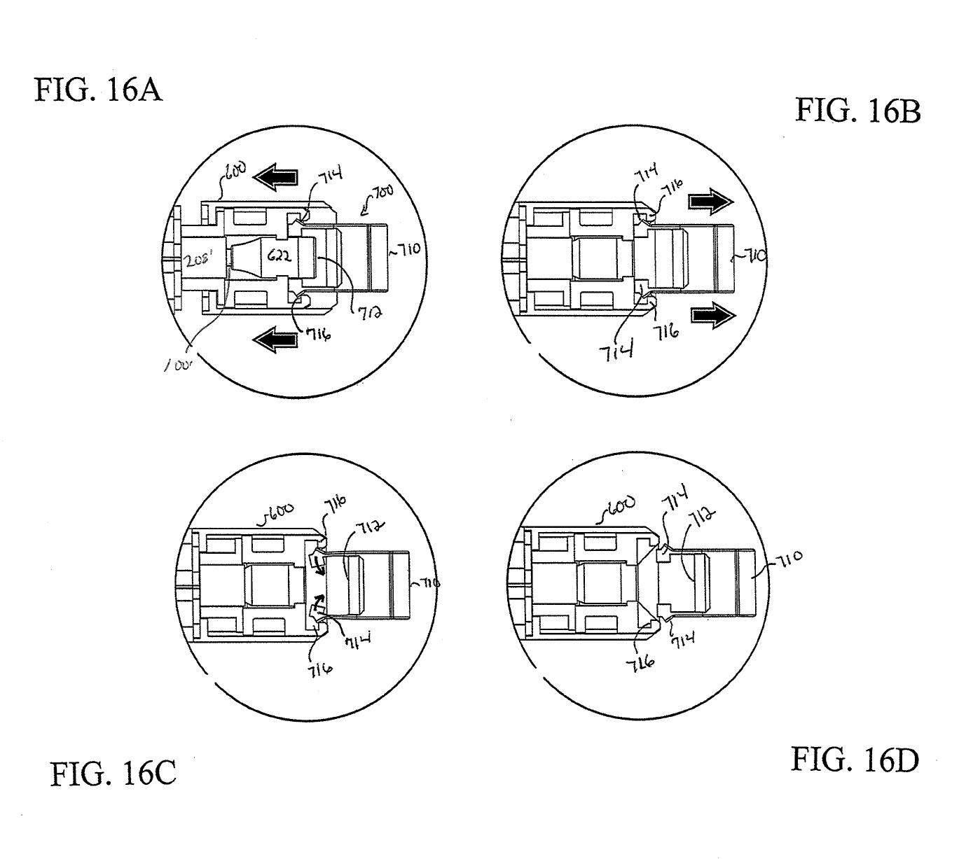

Referring now to FIGS. 16A-16D the operation of the safety cap is show. FIG. 16A show the slidable valve adaptor in the first proximal position prior to being slidably advanced to the distal position. Safety cap 700 is in the “blocking” position in which valve lumen 613 is blocked. Resilient safety cap hooks 714 are resiliently biased in the expanded position as shown in FIG. 16A and engage retaining hooks 716 operably coupled to mating halves 24′, 26′. In this position, the safety cap hooks cannot flex because the valve 622 is in the way. Referring to FIGS. 16B and 16C, as the slidable valve adaptor is slidably advanced to the distal position, the valve 622 moves distally in relation to the retaining hooks 716 and the safety cap hooks 714 as shown in FIG. 16B. In this position, the safety cap hooks are free to flex and therefore if the safety cap is pulled proximally by the user, the safety cap hooks will bend around the retaining hooks as shown in FIG. 16C and the safety cap will be removed as shown in FIG. 16D.

Although the present invention has been described with reference to various aspects of the invention, those of ordinary skill in the art will recognize that changes may be made in form and detail without departing from the spirit and scope of the invention.

We claim:

1. An MR compatible steerable sheath comprising:
a steerable shaft including a proximal end and a deflectable distal tip, said steerable shaft configured to receive first and second longitudinal movement wires operably coupled to said deflectable distal tip, said proximal end slidably receivable within a lumen of a t-valve axel;
a hemostasis valve assembly operably coupled to the proximal end of the steerable shaft;
a slidable valve adaptor operably coupled to said hemostasis valve assembly and configured to be slidably receivable within said lumen of said t-valve axel;
a control handle having a main body configured to receive said valve adaptor and hemostasis valve assembly and said first and second rack screws, said second rack screw including a threaded portion on an outer surface thereof, said steerable shaft extending axially through said control handle;
said first longitudinal movement wire operably coupled to said first rack screw and said second longitudinal movement operably coupled to said second rack screw; and
a rotatable adjustment knob operably engageable with said control handle, said rotatable adjustment knob having an internal threaded portion matingly engageable with the threaded portion of said second rack screw, said rotatable adjustment knob moveable between a first position in which the internal thread is configured to engage the thread on the outer surface of said second rack screw and cause said second rack screw to move proximally to cause proximal longitudinal movement of the second longitudinal movement wire and a second position in which the internal thread is configured to move said second rack screw in a distal direction to release tension on the second longitudinal movement wire,
wherein said valve adaptor is configured to remove said slack from said first and second longitudinal movement wires when slidingly moved to a second position.

2. The MR compatible steerable sheath of claim 1 wherein said valve adaptor is slidably moveable from a first position to said second position, said valve adaptor configured to remove slack from said first and second longitudinal movement wires when said valve adaptor is in the second position.

3. The MR compatible steerable sheath of claim 2 wherein said first position is proximal and said second position is distal.

3. The MR compatible steerable sheath of claim 1 wherein said valve adaptor is slidably moveable between a first position and said second position, said valve adaptor configured to remove slack from said first and second longitudinal movement wires when said valve adaptor is in a second position.

4. The MR compatible steerable sheath of claim 1 further comprising a locking mechanism including a first locking element engageable with a second locking element.

5. The MR compatible steerable sheath of claim 4 wherein said first locking element is a hook and said second locking element is a barb.

6. The MR compatible steerable sheath of claim 4 wherein said first and second locking mechanisms are selected from the group consisting of snap hooks, annular snaps, detents, magnets, living hinge hooks and combinations of the foregoing.

7. The MR compatible steerable sheath of claim 4 wherein said first locking element is positioned on a proximal end of said control handle and said second locking element is positioned on said valve adaptor.

8. The MR compatible steerable sheath of claim 1 further comprising a safety cap engageable with said slidable valve adaptor.

9. The MR compatible steerable sheath of claim 8 wherein said safety cap further comprises at least one resilient safety cap hook.

10. The MR compatible steerable sheath of claim 9 wherein said at least one safety cap hook is resiliently biased in the expanded position.

11. The MR compatible steerable sheath of claim 10 wherein said at least one safety cap hook is configured to engage at least one retaining hook on said valve adaptor.

12. The MR compatible steerable sheath of claim 11 wherein said safety cap is moveable from a first position to a locked second position.

13. The MR compatible steerable sheath of claim 12 wherein said first position is distal and second position is proximal.

14. The MR compatible steerable sheath of claim 13 wherein when said safety cap is in said second position the safety cap is configured to be removable from said valve adaptor.

15. The MR compatible steerable sheath of claim 9 wherein said safety valve further comprises a finger-graspable portion.

16. The MR compatible steerable sheath of claim 9 further comprising a blocking element configured to block said hemostasis valve lumen.

17. An MR compatible steerable sheath comprising:
a steerable shaft including a proximal end and a deflectable distal tip, said steerable sheath configured to receive first and second longitudinal movement wires operably coupled to said deflectable distal tip;
a hemostasis valve assembly operably coupled to the proximal end of the steerable shaft;
a slidable valve adaptor operably coupled to said hemostasis valve assembly;
a control handle having a main body configured to slidably receive said valve adaptor and said hemostasis valve assembly and configured to receive first and second rack screws, said second rack screw including a threaded portion on an outer surface thereof, said steerable shaft extending axially through said control handle;
said first longitudinal movement wire operably coupled to said first rack screw and said second longitudinal movement operably coupled to said second rack screw; and
a rotatable adjustment knob operably engageable with said control handle and moveable between a first position which causes said second rack screw to move proximally to cause proximal longitudinal movement of the second longitudinal movement wire and a second position in which the second rack screw moves in a distal direction to release tension on the second longitudinal movement wire,
wherein said slidable valve adaptor is configured to remove slack from said first and second longitudinal movement wires.

18. The MR compatible deflectable sheath of claim 17 wherein said valve adaptor is slidably moveable from a first position to a locked second position.

19. The MR compatible deflectable sheath of claim 17 wherein said first position is proximal and said second position is distal.

20. The MR compatible deflectable sheath of claim 17 wherein said valve adaptor is slidably moveable between a first position and a second position.

21. The MR compatible deflectable sheath of claim 18 wherein the distance between the first and second positions is approximately 0.250″ or greater.

22. The MR compatible deflectable sheath of claim 20 wherein the distance between the first and second positions is approximately 0.250″ or greater.
